(19) United States
(12) Patent Application Publication
(10) Pub. No.: US 2018/0263597 A1
(43) Pub. Date: Sep. 20, 2018

(54) DEVICE FOR FIXATING A MEDICAL INSTRUMENT

(71) Applicant: MEDACC HOLDING B.V., Eindhoven (NL)

(72) Inventors: Benjamin Ching Yi TCHANG, Bavel (NL); Victor Barend Hendrik DONKER, Eindhoven (NL); Jori Marcellinus Nicole VERBEEK, Eindhoven (NL)

(21) Appl. No.: 15/762,134

(22) PCT Filed: Sep. 7, 2016
(86) PCT No.: PCT/NL2016/050622

§ 371 Date: Mar. 22, 2018

(30) Foreign Application Priority Data
Sep. 24, 2015 (NL) 1041488

(51) Int. Cl. A61B 8/00; G10K 11/00; G10K 11/35

(52) U.S. Cl. CPC A61B 8/4227; A61B 8/4281; G10K 11/355; A61B 8/4477; G10K 11/004; A61B 8/4411

(57) ABSTRACT

The present invention relates to a device for holding an interchangeable ultrasound probe for medical application. The device comprises a base comprising a fixation means arranged for fixating the device at a surface contact area of the device to a position on a patient's skin; and a base module arranged for being connected to the base and arranged for holding the ultrasound probe. The base module further comprises adjusting means arranged for adjusting the position of the ultrasound probe. The adjusting means comprises a locking mechanism arranged for fixating the adjusted position of the ultrasound probe. The base module comprises a receiver arranged for receiving and retaining the ultrasound probe, which receiver is customizable to the ultrasound probe. The invention further relates to a method.

### TECHNICAL FIELD

The invention relates to a device for positioning of a medical instrument. In particularly, the invention relates to a device for adjusting the position of a medical imaging instrument, such as an ultrasound probe, in a number of rotational and translational axes. More particular, the invention relates to a device for positioning, fixating and/or stabilizing said probe on a patient's body.

### BACKGROUND

Medical imaging instruments are frequently used by medical doctors and other medical professionals (hereinafter both referred to as “physician”) to conduct non-invasive examination of humans and animals. For the purpose of this type of imaging a so called ultrasound transducer or probe (hereinafter abbreviated as “probe”) is often used. A probe may be effectively used to examine internal tissue that is not readily examined using normal visual and tactile examination. Kidney stones, tumors, cysts, etc. are all amenable to examination using these medical imaging instruments. Examples of these medical instruments are scanning instruments for creating images, based on e.g. Computed Tomography (CT), Magnetic Resonance Imaging (MRI), Positron Emission Tomography (PET) or Single-Photon Emission Computerized Tomography (SPECT). In addition, medical imaging instruments are well suited to examination of a growing fetus and to determination of the health of the fetus and to making medical diagnosis ‘to improve the fetus’ health. The physician holds the probe as close as possible to the patient's skin at the area that is to be studied or measured.

Traditionally, the position of the probe is controlled by holding the probe in one or both of his hands (whenever “he” or “his” is written, one should also read “she” or “her”). In this manner, the physician manually guides the instrument over the body. Such methods are suitable for many medical procedures, but also pose significant shortcomings in other procedures. One problem is that some studies require a long-term measurement, in which the laboratory physician sits in the same position for a long time, while the ultrasound transducer is held against the body of the patient by hand. Another problem associated with manually holding the probe is that the probe can fully occupy one hand of the medical practitioner, thereby making it more difficult to perform ancillary medical procedures, such as removal of a biopsy sample, or even the taking of notes or. Moreover, manually holding the probe can create physical complaints for the practitioner which are related to adverse ergonomics. Often, current solutions even require an extra person to help. Another significant problem associated with holding the probe is that it can be difficult to hold the probe steady, for example because of a trembling hand, and thus it is difficult to “fine tune” the probe and direct the imaging field to precise locations in a patient at the same time. This fine tuning of the probe location can be particularly important when very localized tissue sampling or medical procedures are being performed, such as during surgical procedures.

One particular problem occurs when measurements need to take place while the patient is exposed to dynamic loads. A physician needs to measure for example an area of a patient's leg while the patient is moving his leg. It may for example be necessary to measure blood flow through the patient's leg arteries while the patient is cycling on an exercise bike.

Several devices have been disclosed which facilitate the ultrasound imaging procedures by providing assemblies with integrated probe of special holders for either holding the probe in a static position, or for easily measuring a patient's body on various positions dynamically. Examples of current devices are described below.

A single purpose solution is disclosed in European patent application EP2666414A1 by Zijlstra, wherein the invention relates to an ultrasound device, in particular an ultrasound device that is designed for vascular imaging, especially for guiding vascular puncture. It comprises an ultrasound imaging probe that can be stabilized at the point of puncture by integrated straps or by a handle designed to rest the probe like a stethoscope on the skin as to enable puncture at suitable spots of the human body to gain access to the vascular system. In this way the major problem for lesser trained hands to combine the plane of imaging of the probe with the imaging plane of the needle is overcome. The manual puncture process is aided by the ultrasound device by showing an image of the vessel to be punctured both in a longitudinal and an axial way, which together with the stabilized position of the device, enables the manual puncture to be as accurate as possible.

USA patent application US2011/0022034A1 by Civco Medical Instruments Co, Inc. discloses a system for positioning a laparoscopic device including a curvilinear articulating arm and a holder. The holder has at least two rotational regions and a clamping portion for receiving the laparoscopic device, and the holder is coupled to the curvilinear articulating arm. The at least two rotational regions are permitted to articulate. In addition, a method of positioning a laparoscopic device in a skin port of a mammal includes: securing the laparoscopic device to a holder having at least two rotational joints; coupling the holder to a curvilinear articulating arm; disposing the laparoscopic device partially within the skin port; positioning the laparoscopic device by selectively articulating the curvilinear articulating arm and selectively rotating portions of the holder with respect to one another.

U.S. Pat. No. 6,301,989 B1 discloses a positioning apparatus for adjusting the position of medical imaging instruments, such as ultrasound probes. The preferred implementation of the apparatus includes controls for translational movement along three axes; and controls for rotational movement around three axes. In certain implementations, the rotational movement is around a point coincident with the area of image capture.

European patent application EP0331348A1 by Hon et al. discloses a holder for attaching an ultrasonic transducer to the body of a patient, particularly to obtain the fetal heart rate in a pregnant patient, that does not require the use of belts. The holder comprises a flexible disc having a central support member with a central opening for receiving an ultrasonic transducer. Grasping means in the form of two screws are provided in the side wall of the support member for engaging a transducer to lock it in position. The holder is adhesively attached to the patient and permits the ultrasound transducer to be held in a desired orientation.

A disadvantage of the current art solutions is that their purpose only suits particular medical instruments which can only measure in particular situations and/or positions of the patient's body.

### DISCLOSURE OF INVENTION

It is an object of the present invention to provide a holder which is suitable for a broad range of medical instruments. It is a further object of the invention to provide means for adjusting the position of the medical instrument. It is yet a further object of the invention to provide a reliable fixation of the holder to a patient's body. It is yet a further object of the present invention to provide a device which fulfills the above objects with the least possible complexity and maximum ease of use and reliability.

The object is realized by the invented probe holder, which ensures that there is a correct fixation of the probe at a specific place on the body. By employing a generic ring with an interchangeable movable module having a receiver for holding a particular instrument, and/or a standard movable module with an interchangeable receiver for a particular instrument, the device may be used for a wide variety of instruments, without the need to adapt or replace the complete device. This saves costs and makes it possible to leave the device at a fixated position while replacing the instrument by another instrument. This in turn causes less inconvenience for both patient and physician. The interchangeability of modules and/or receivers is facilitated by the invented modular construction, which allows quick and easy replacement.

Advantageously, the device according to the current disclosure provides for a generic receiver being customizable to the size and/or shape of an ultrasound probe. In an advantageous embodiment, the device provides for a modular receiver and/or receiver module which can be replaced by another receiver and/or receiver module which is selected based on the size and/or shape of an ultrasound probe to be received and retained by said selected receiver.

The invention comprises the following clauses.

1. A device for holding an instrument for medical application comprising:
a base comprising a fixation means arranged for fixating the device at a surface contact area of the device to a position on a patient's skin;
a module arranged for being connected to the base and arranged for holding the instrument;
the module further comprising adjusting means arranged for adjusting the position of the instrument,
the adjusting means comprising a locking mechanism arranged for fixating the adjusted position of the instrument,

characterized in that,

the module comprises a receiver arranged for receiving and retaining the instrument wherein the instrument comprises an interchangeable instrument.

2. The device according to clause 1, characterized in that the module is arranged for being displaced in a rotational direction from a first rotated position to a second rotated position in the plane of the surface area.

3. The device according to any one of the preceding clauses, characterized in that the module is arranged for being displaced in fixed steps.

4. The device according to any one of the preceding clauses, characterized in that the base comprises a ring having a first raised wall and the module comprises a ring having a second raised wall.

5. The device according to any one of the preceding clauses, characterized in that the module is arranged for being tilted from a first tilted position to a second tilted position compared to the plane of rotation.

6. The device according to any one of the preceding clauses, characterized in that the locking mechanism comprises a configuration of the group of configurations comprising:
a configuration for locking the module in a rotated position, wherein multiple inner longitudinal recesses are comprised in the first wall, said recesses positioned substantially perpendicular to the plane of the surface area, and wherein said recesses are arranged for receiving at least one protrusion arranged on the outside of the second wall;
a configuration for locking the module in a rotated and/or tilted position, wherein the module comprises a locking element, such as a friction element, a bolt or a screw which is arranged for locking and/or unlocking rotation of the module by manipulating the locking element,
a configuration for locking the module in a rotated and/or tilted position, wherein the module comprises a friction element exerting a default level of friction force for sufficiently holding the module in a first tilted position, and which may be displaced to a second tilted position by manual power overcoming the default level of friction force.

7. The device according to clause 6, characterized in that the module comprises a rotatably connected tilt member, said tilt member comprising the at least one protrusion.

8. The device according to any one of the preceding clauses, characterized in that the receiver comprises a fitting arranged for being fit into the receiver module and which comprises a through hole arranged for inserting or removing the instrument.

9. The device according to any one of the preceding clauses, characterized in that the material surrounding the through hole of the fitting comprises a resilient material such as silicone, silicone rubber, or rubber.

10. The device according to any one of the preceding clauses, characterized in that the fixation means comprises at least one strap, said strap comprising a strap of the group comprising:
an elastic band comprised of at least one part for surrounding a body part of the patient and arranged for being fastened to the device or to another part of the band.
a non-elastic strap comprised of at least one part for surrounding a body part of the patient and arranged for being fastened and tightened to the device or to another part of the strap.

In an advantageous embodiment, a connection can be employed which enables relative angular movement between two components. Such a connection is preferably arranged between the base and the base module. As a consequence the receiver module and/or receiver, held by the base module, can then also be orientated and fixed appropriately. For this purpose, a ball and socket connection can be employed for facilitating the relative angular movement between the two components, namely the base and the base module. The base comprises a substantially hemispherical depression in which the spherical shaped base module can be received. The shape of the base module is complementary to the spherical surface of the base. The base encloses a substantial portion of the base module. Furthermore, the base module is in rotatable engagement with the base. Significant ranges of motion of the base module with respect to the base can be achieved. Due to this, the probe can be easily orientated with respect to the patient's body. Advantageously, the ball and socket connection (cf. ‘ball joint’), comprising a base (cf. socket) and a base module (cf. ball) movable mounted in the base, may further comprise locking means for fixing the base module relative to the base. Such locking may also be achieved by sufficient friction between the elements forming the ball and socket connection. Various alternative ball in socket embodiments are possible. In an example, the base module can have spherical lateral sides and a substantially flat upper and lower portion. Also the base may comprise a substantially flat upper portion. In this way, handling of the probe and changing the probe or modular receiving module can be facilitated, because there may be more space for manually handling the probe.

Optionally, the base is arranged to be displaceable with respect to the base module such that the relative displacement between the base and the base module in a longitudinal direction of the base and/or base module is adjustable. In an example, the longitudinal direction corresponds to a longitudinal direction of the device. In an advantageous embodiment, the base is displaceable with respect to at least one of the receiver module, receiver or base module in a longitudinal direction of the device. The base can also be movably connected to a base holder arranged for holding the base. Advantageously, the surface contact area can provide a contact interface with a patient's body.

The device may comprise a superior portion configured to receive an ultrasound probe, an inferior portion opposite to the superior portion configured to be seated on a patient's body, a lateral portion defining a length or height of the device between the superior portion and the inferior portion. A longitudinal direction may correspond to a longitudinal path between the superior portion and inferior portion. This path can be an axially extending line from the superior portion to the inferior portion towards the patient's body, e.g. when the device is secured to the patient's body. Such axially extending line can be tilted with respect to a patient's body. For example, the orientation of the base module can be rotatably adjusted so as to be able to adjust the orientation of the ultrasound probe with respect to the patient's body. However, as a result, a longitudinal direction or path of the base module can also be tilted with respect to the patient's body. Displacement along a longitudinal direction of a member of the device towards or away from a patient's body, during use, allows the adjustment of the height/depth of the receiver and hence the ultrasound probe with respect to the patient's body.

Optionally, the distance of the receiver to the surface contact area is adjustable in a longitudinal direction of the device. For this purpose, the receiver can be arranged to be displaceable with respect to the surface contact area so that a distance of said receiver to said surface contact area is adjustable. In an example, the distance is adjusted by a translation of the receiver. The device may also be configured to employ other moving mechanisms such as a twisting (rotation and translation) movement. The displacement in the longitudinal direction can result in a height adjustment of the receiver in relation to the base. In an advantageous embodiment, the receiver is slidably movable with respect to the base. Height adjustment of the receiver by means of axial displacement relative to the surface contact area may ensure that the receiver can be angularly movable and longitudinally displaceable relative to the surface contact area so that different positional orientation of the probe with respect to a patient's body can be obtained. In an advantageous embodiment, the receiver is longitudinally displaceable along a longitudinal axis of the device. The receiver can be arranged to longitudinally receive a probe so that a head of the probe faces the surface contact area and thus the patient's skin when in use.

Advantageously the receiver can be vertically movable up and down toward and away from the surface contact area so that the contact pressure of the head of the probe exerted on the patient's skin can be adjusted by adjustment of the height of the probe or distance of the probe with respect to the patient's skin.

Optionally, the base module is movably connected to a base so that the distance between the base module relative to the surface contact area is adjustable. In this way, the receiver can be moved by relative movement of the base module with respect to the base so that the distance between the receiver and the surface contact area can be adjusted. The receiver may be moved directly or by means of moving of the transducer. Alternatively, the transducer may be held in the receiver in such a manner as to allow relative displacement between the transducer and the receiver for adjusting height of the probe. Preferably, the base module is mounted in the base in such a way that the orientation of the base module with respect to the base is adjustable. Consequently, the base module and thus the receiver can be orientable and/or displaceable relative to the surface contact area.

In an advantageous embodiment, the receiver is rotationally movable and longitudinally displaceable relative to the surface contact area.

In an example, the distance of the receiver to the surface contact area is slidably adjustable, wherein the receiver is guided in longitudinally sliding fashion on the base so as to adjust the distance between the receiver and the surface contact area. For this purpose, the receiver can be longitudinally displaceable mounted in the base module.

Optionally, a height locking means is arranged for locking the longitudinal distance of the receiver with respect to the surface contact area. In this way, an adjusted distance between the receiver and the surface contact area can be set and locked at a desired height. By means of height adjustment locking means, an adjusted distance can be locked in a plurality of positions arranged between a retracted position and an extended position of the receiver. The device can be arranged to push the head of a received probe against the patient's skin when moving the receiver from the retracted position to the extended position.

Optionally, the height locking means is configured to provide discrete locking positions in which the distance between the receiver and the surface contact area is fixed. An adjustment of the distance between the receiver and the surface contact area allows a height adjustment of the receiver relative to the surface contact area. The receiver can be fixed at the adjusted distance/height by the height locking means so that the contact pressure or pressing force of the ultrasound probe on a patient's skin can be adjusted. This allows the operator of the ultrasound prove to obtain more control over the measurements. A distance between the receiver and the surface contact area can be seen as a height of the receiver.

Optionally, the device further comprises supporting means arranged for providing a contact interface between the device and a patient's skin, wherein the supporting means comprises at least three contact legs. Arranging three legs can be advantageously for the stability of the support provided by the supporting means. In an advantageous embodiment, the legs have rounded distal ends which are configured to contact the patient's skin. This can further improve the stability, especially on non-planar three-dimensional patient body surfaces and/or flexible patient body surfaces. Moreover, the contact area can be enlarged and the forces exerted on the device may be more appropriately distributed over said contact area.

Optionally, the supporting means comprise contact legs having one or more removable and/or disposable portions. Advantageously, the risk of (medical) contamination of the device can be at least partially reduced in this way. Instead of or in addition to removable or disposable portions of the contact legs, suitably shaped covers or sleeves may be used to cover distal ends of the contact legs, i.e. those portions making contact with a patient's skin. Such sleeves or covers may then be made disposable. In an embodiment, the contact legs are connected to or integrated with the base.

Optionally, the contact legs comprise a base and a distal end, the base arranged for being coupled with the device, the distal end having a surface contact area configured for being in contact with a patient's skin so as to provide support for the device, wherein the distal end is pivotally connected with the base. Such a pivotally connected distal end can be more adaptable to a non-planar three-dimensional patient body surface. Consequently, in this way, the device may be better secured to the patient's body.

Optionally, the device further comprises a configurable fastening member providing a plurality of selective fastening positions at which one or more fastening means can be attached. In this way, more freedom is provided in securing the device on a patient's body.

The invention further relates to a method for holding an interchangeable ultrasound probe for medical application, the method comprising: selecting an ultrasound probe with a custom size and shape, placing the ultrasound probe in the receiver, wherein the receiver is adjusted or corresponds to at least one of the size or the shape of the selected ultrasound probe such as to be customized for retaining the probe.

Optionally, the base module and/or receiver are modular and arranged for being interchangeable for a particular ultrasound probe, wherein customizing the receiver comprises replacing the base module and/or receiver so as to adapt to at least one of the size or the shape of the ultrasound probe. Such a modular design of the device can substantially enhance the versatility of the device.

Optionally, the method further comprises securing the device on a patient's body and adjusting the height of the receiver with respect to the patient's skin In this way, the probe-skin interaction can be adjusted. Also the pushing force or pressure exerted by the probe onto the patient's body can be adjusted.

### BRIEF DESCRIPTION OF THE DRAWINGS

The figures show views of embodiments in accordance with the present invention.

FIG. 1 shows a perspective view of an example embodiment of the invented device.

FIG. 2 shows a perspective view of an example embodiment of the invented device with an example of an inserted medical instrument held by the device.

FIG. 3 shows a perspective view of a detail of the present invention.

FIG. 4 shows a perspective view of a detail of the present invention.

FIG. 5 shows an overview of a situation wherein the invented device is attached to a patient.

FIGS. 6A & 6B show an embodiment of the present invention.

FIG. 7. shows a cross-section of an embodiment of the present invention.

FIG. 8. shows a cross-section of an embodiment of the present invention.

FIG. 9A-9E. show an embodiment of the present invention.

FIG. 10A-10B. show a schematic cross-section of an embodiment of the present invention.

FIG. 11A-11C. show a schematic cross-section of an embodiment of the present invention.

### DETAILED DESCRIPTION

The invention is now described by the following aspects and embodiments, with reference to the figures.

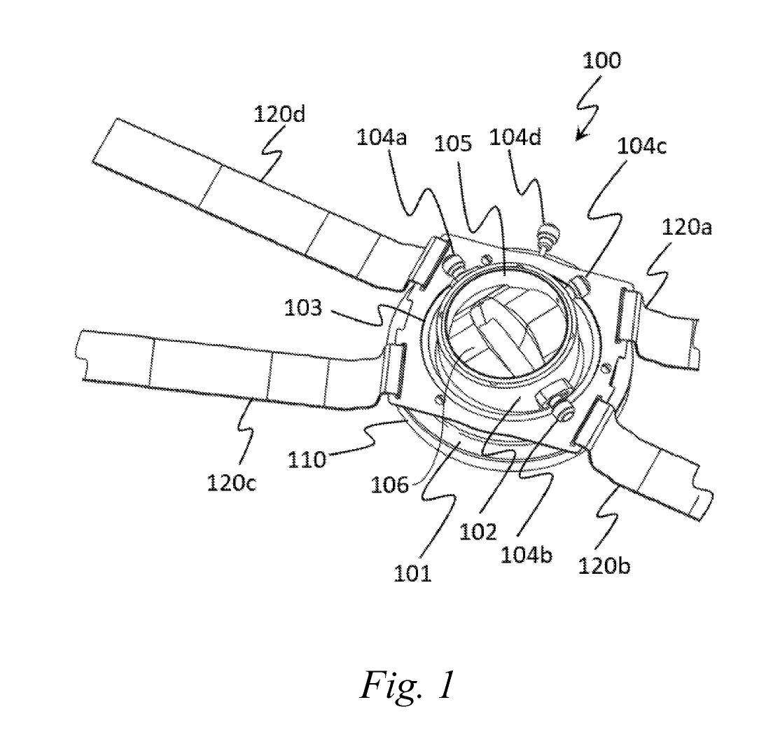

FIG. 1 shows a perspective view of an example embodiment of the invented device.

The device comprises several parts. A first part comprises a base 101, which is arranged for being fixated on a patient's body. For this purpose fastening means 120a,b,c,d are attached to the base 101. These fastening means 120a,b,c,d are preferably elastic bands or straps. The base 101 is arranged for supporting the second part, which comprises a base module 102 arranged for being movable in multiple directions within the base 101. Base 101 comprises a generic ring 103, which is movably connected to the base 101. Ring 103 may be rotated around a virtual central axis. Ring 103 holds base module 102 which may be tilted around an axis perpendicular or any angle to the virtual central axis. Ring 103 and/or base 101 may comprise locking means 104a,b,c,d for securing ring 103 in a preferred position.

Base module 102 comprises a receiver module 105 which is arranged for holding a customized or customizable adapter or receiver 106 for receiving and retaining a medical instrument such as an ultrasound probe. Preferably receiver 106 is made of a resilient material such as silicone or silicone rubber which enables a tight fit of the instrument. Receiver 106 may also comprise softer materials. The materials may for example be manufactured using vacuum press. Receiver 106 may also comprise a part which may be inflated in order to secure the medical instrument. Another way of securing may involve the use clips or other locking mechanisms. The invented device 100 enables a stable fixation of medical measurement instruments. Instead of a measuring instrument also other instruments may be inserted, for example a biopsy needle, or an endoscope. Alternatively the device may be enhanced with an extra slot or groove or other guidance system which enable the insertion of a needle along with the use of a medical instrument inserted in receiver 106 To facilitate the stable use of a needle and another instrument at the same time, the device may incorporate a connector (not shown) to connect other equipment, for example for holding and guiding a needle, to the invented device. The connector may even be arranged for connecting a second invented device. In this way an almost limitless extension of functionality and numbers of connected devices may be realized.

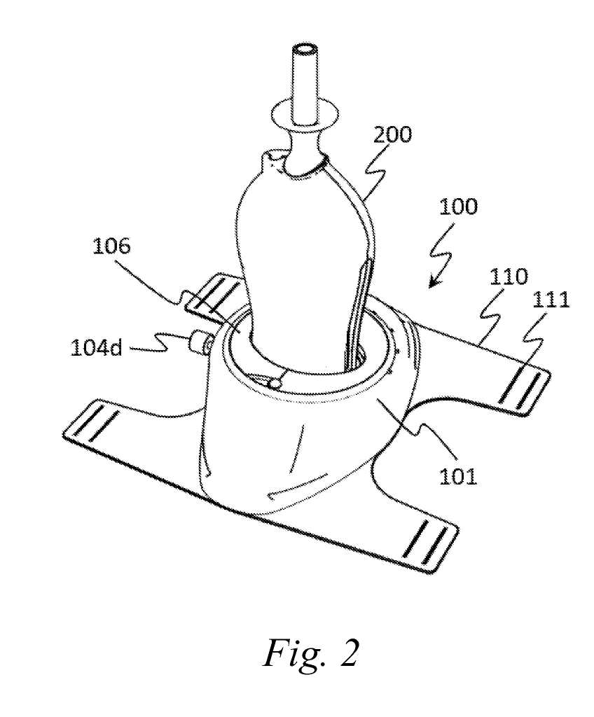

FIG. 2 shows an example embodiment of the invented device 100 with an example of an inserted medical instrument 200 held by the device 100. Base 101 comprises a contact layer 110 which is arranged for making contact with a body part of a patient. In this example the contact layer 110 also comprises slots for attaching one or more straps. These slots may be situated on other locations of the device 100 though. The instrument 200 is held tight by receiver 106, which may be locked by locking means 104d. The surface contact area can be formed by the contact layer 110. The surface contact area can comprise a portion of the device which comes into contact with the body during use.

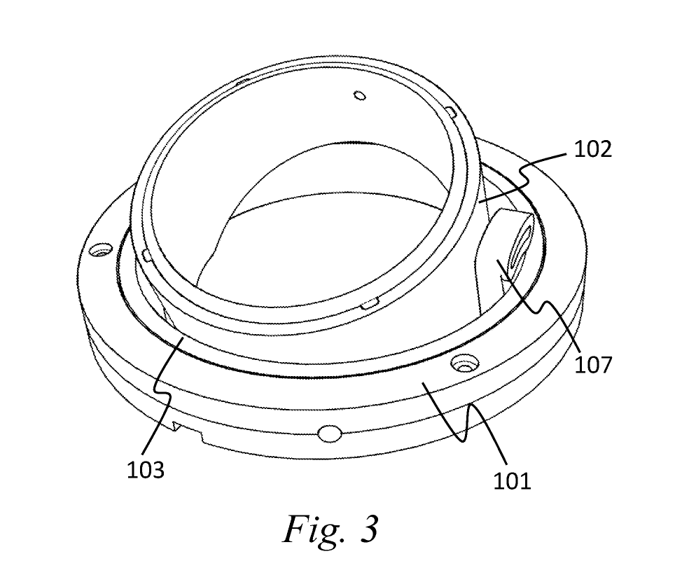

FIG. 3 shows a perspective view of a detail of the present invention 100. Base 101 comprises generic ring 103. Base module 102 is rotational by rotating ring 103 within base 101 and is tiltable within ring 103. The figure shows base module 102 in a tilted position. Typical the tilt comprises around 20 degrees, but larger tilting angles are possible depending amongst others on the height of the axis 107 and the room below base module 102. The bottom of base module 102 may comprise slanted sides in order to allow larger tilting angles. By tilting and rotating the base module 102 the most suitable position for the medical instrument may be chosen by the user.

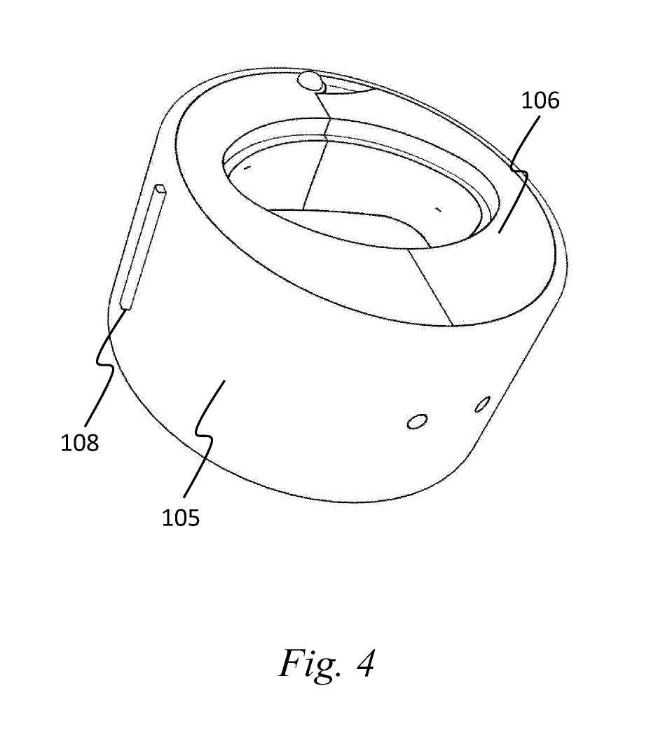

FIG. 4 shows a perspective view of an example detail of the present invention 100. Base module 102 comprises receiver 106. Base module 102 also comprises in this example a configuration for locking the module in a rotated position, wherein multiple inner longitudinal recesses are comprised in inner wall of the generic ring 103. These recesses are positioned substantially perpendicular to the plane of the surface area, and arranged for receiving at least one protrusion 108 arranged on the outside of the wall of base module 102. By removing base module 102 from the ring 103 and rotating base module 102, base module 102 may again be inserted into ring 103 at a different rotated position.

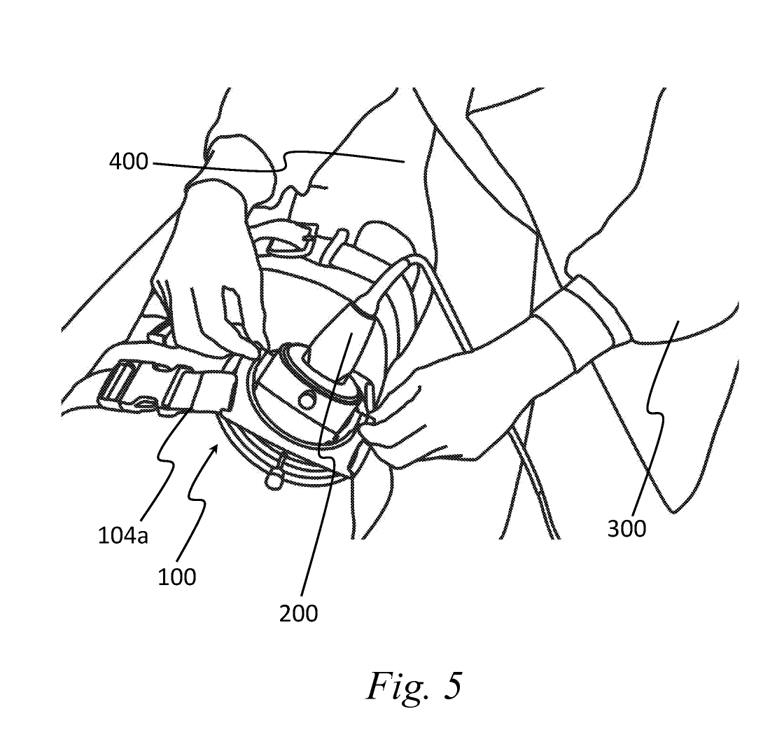

FIG. 5 shows an overview of a situation wherein the invented device 100 is attached to a patient 400, for example to a patient's chest, but other parts may be possible as well. An example medical instrument 200 is inserted to and held by device 100. A physician or any other user 300 has fixated the device 100 to patient 400 by means of straps 104a,b,c,d. or (elastic) bands wherein the pressure is distributed evenly over the surface of the skin. For each probe a specific fit (receiver) may be available, the probe is securely fastened inside the module. The module with the probe can move freely in the two-dimensional plane, so that the medical instrument may rotate 360 degrees around and tilted in both directions. Finally the base module 102 is locked and all is in place to start for example measurements with the medical instrument 200.

Application areas of the invention comprise the following:

Firstly, an ultrasound medical instrument may be retained by the invented device. The ultrasound medical instrument may be used for:
contrast administration;
ultrasound therapy, i.e. physiotherapy;
operations while retaining the ultrasound transducer must be prolonged;
measurements in which subjects have to sit still for a long time;
dynamic measurements, i.e. muscle measurements, in which the patient moves during the measurement;
diagnosis of a particular disease, which is examined with ultrasound, or which is in particular detectable during an exercise or stress test;
removal of fat from the abdomen or buttocks where it is important that the ultrasound transducer is stabilized at a particular place;
rehabilitation therapy where the patient has to do certain exercises over an extended period, whereby ultrasound measurements need to be done to see whether a drug is working. For example by determining the effect of the drug on freedom of movement of limbs or joints;
diagnosis of patients who have lost a certain degree of freedom of movement
examination of patients with knee, bone or shoulder Injury injuries who have to make particular movements, while a physiotherapist looks at groups of muscles that influence the freedom of movement;

Secondly an MRI or CT device may be retained by the invented device. These types of devices may be used for:
contrast administration
measurements whereby at a certain location on the patient's body need to be applied cold or hot compression
measurements whereby certain sensors have to be retained in certain places.

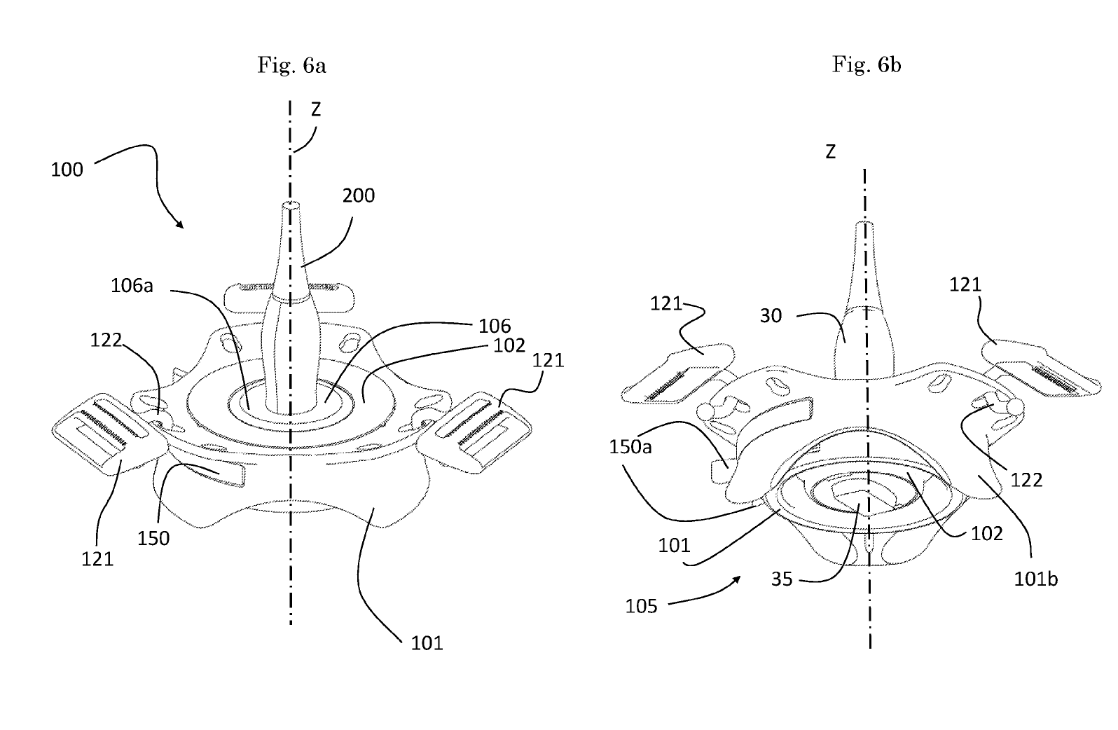

FIGS. 6a and 6b show an embodiment of the device 100, wherein the receiver is arranged to longitudinally receive a probe 200. The probe 200 can be inserted into receiver 106, so that a head 35 of the probe can be directed towards a patient's body so as to be in contact with the patient's skin, when in use. In this advantageous embodiment, the receiver 106 (and receiver module 105) are displaceable along a longitudinal axis Z of the device 100 (vertical in the figure). The distance of the receiver 106 to the surface contact area 101b is adjustable in a longitudinal direction along the longitudinal axis Z of the device. The receiver 106 can be translated with respect to the base 101 so as to adjust the distance of the receiver 106 with respect to the surface contact area 101b. This distance can be seen as a height of the receiver 106 in relation to the base 101. The receiver 106 is rotationally movable with respect to the base 101 so as to enable orientation of the probe 200 with respect to the surface contact area. The base module 102 and thus the receiver 106 which is mounted on the base module 102, is further also axially displaceable, i.e. vertically movable up and down toward and away from the surface contact area, with respect to the base 101 so as to adjust the distance of the receiver 106 with respect to the surface contact area (i.e. height). In this way, if a probe is held by the receiver 106, the contact pressure of the head 35 of the probe 200 exerted on a patient's body or skin can be adjusted by adjustment of this distance or height. Hence, the ultrasound probe 200, held by the receiver 106, can be orientated, tilted, rotated and translated with respect to the surface contact area, and thus the patient's skin when in use. The surface contact area can be seen by one or more regions arranged for being brought in contact with the patient when mounting the device on a desired location of the patient's body.

The base module 102 carrying the receiver module 105 and receiver 106 is movably connected to the base 101, wherein the relative orientation of the base module 102 with respect to the base 101 can be adjusted and fixed at a desired adjusted orientation. Furthermore, the device comprises height locking means 150 arranged for locking the relative longitudinal displacement of the base module 102 with respect to the base 101, and hence the longitudinal distance of the base 101 with respect to the surface contact area. Since the base module 102 is holding the receiving module 105 and receiver 106, an adjusted distance between the receiver 106 and the surface contact area can be set and locked at a desired height. In the shown embodiment, the height locking means 150 comprises a handle 150a for releasing the locking means 150.

In contrast to integral and/or fixed-design devices, the receiver 106 is, advantageously, customizable to at least a part of the shape of the used ultrasound probe, and may therefore also be compatible for use with various other ultrasound probe types and models. Moreover, the receiver is typically subject to much wear and tear since the ultrasound probe 200 is removably connectable to the receiver 106, making replacement of a removable and/or disposable receiver module 105 and/or receiver 106 beneficial. As a result, the replacement and operating costs can be significantly reduced using a replaceable receiver. Furthermore, the risk of contaminating the device may be reduced.

The receiver module 105 and/or receiver 106 may be configured to enable coupling, connecting and/or mounting of an ultrasound probe thereon. The device 100 can facilitate both periodic and continuous sensing by the ultrasound probe. The receiver 106 comprises a fitting 106a arranged for being fit into the receiver module 105 or module 106 and which comprises an insertion opening or through hole arranged for inserting or removing the ultrasound probe 200. The insertion opening can define a passage-way for the ultrasound probe through the device, so that a contacting interface between the head (distal end) of the ultrasound probe and a patient's skin can be established, in use. The receiver 106 can thus enable elastic fixation of the ultrasound probe 200. The material surrounding the insertion opening or through hole of the fitting 106a comprises a resilient material such as silicone, silicone rubber, or rubber. The resilient material may for example be an elastomer material such as a medical grade silicone polymer.

The fitting 106a may comprise a resilient inner member conforming in shape to at least one custom shape of an ultrasound probe 200, so that the ultrasound probe 200 can be held snugly in place. Advantageously, the fitting 106a is replaceable and can be selected in accordance with the used ultrasound probe 200 so as to allow a tight fit for a plurality of (varying) ultrasound probe models while using the same device 100. The fitting 106a can be custom-molded for one or more ultrasound probe models. In an embodiment, the fitting comprises at least one conforming portion, such as an elastic rubber-like portion, provided with an insertion opening for receiving the ultrasound probe 200. The elastic fitting may allow introduction of an ultrasound probe 200 by pushing said probe in the insertion opening. In this way, adjustment of the receiver, for achieving appropriate clamping, may not be required. Also, the fitting 106a can be stretched to wrap around the ultrasound probe 200 so as to tightly grab said probe 200. Advantageously, the fitting can be replaced at a reasonable cost.

The elastic properties of the material of the fitting 106a and the shape of the fitting can be utilized in that the fitting envelopes and/or hugs at least part of the used ultrasound probe 200. The fitting 106a can be stretched and compressed so that friction mounting of said probe can be enabled. Preferably, the elastic portion of the fitting 106a at least partially envelopes the ultrasound probe 200 at least partially in a form-fitting fashion.

In an advantageous embodiment, the insertion opening in the rubber-like elastic portion of the fitting 106a is smaller than the largest cross-sectional dimension of the ultrasound probe as viewed in a plane perpendicular to the direction of insertion of the ultrasound probe 200 into the insertion opening. As a result, the fitting can, when the ultrasound probe is inserted into the fitting, at least partially close over the ultrasound probe. The receiver module 105 may be designed as an integral, single unit incorporating several sub-units, or it may consist of two or more parts, encompassing at least an adapter part for receiving and holding a removable ultrasound probe. Advantageously, the receiver module 105 is modular and selected and connected and/or coupled with the device, depending on the ultrasound probe to be used. In an example, the fitting may comprise an outer surface including a sterilizing substance.

The fitting 106a thus provides an elastic holder which can wrap around and substantially surround a portion of the sidewalls of the ultrasound probe 200, so as to form a tight encasement around the ultrasound probe 200. Such an elastic self-wrapped holder allows easy and convenient attachment of an ultrasound probe to the receiver 106 without the use of any specialized tools, thereby saving time and effort for the user of the device 100. Furthermore, such elastic self-wrapped holder may prevent or reduce the risk of damaging the ultrasound probe 200 during use and handling of the device 100 and probe 200, while the probe can be firmly secured. The elasticity of the material of the fitting 106a for the ultrasound probe 200 can conform to ultrasound probes 200 of a plurality of sizes and shapes, thereby firmly gripping and tightly fitting the probe 200.

The elastic material can be stretched securely wrapping around the probe. Because of its stretchable attribute, the fitting may not be limited to one particular ultrasound probe model. A plurality of ultrasound probes having acceptable variations in dimensions and shapes can be used with a same fitting. In an embodiment, the removable customizable receiver module 105 and/or receiver 106 is single-use and/or multiple-use disposable. Due to the modular design of the device 100, a fitting 106a having different sizes and shapes can be selected and used in the device 100 to optimally accommodate the dimensions of one or more selected ultrasound probes 200. In an embodiment, the fitting is designed for a particular individual ultrasound probe 200 having a specific shape and size.

The fitting 106a can induce an elastic clamping force to maintain relative orientation between the receiver 106 and the inserted ultrasound probe 200 while being able to handle external loads exerted on said probe without substantial change of the selected relative orientation, for example as a result of the probe head pushing against a patient's body, pressure generated by orientating the probe, handling forces, patient-induced forces, vibrational forces, shock forces, gravity, etc.

In an example, the ultrasound probe 200 is inserted in the device 100 subsequent to securing the device 100 to the skin of the patient. In this way, different probes 200 can be used on a same location, with a same orientation and same insertion depth, as the probes 200 can be inserted and removed from the device with the device 100 being secured to a patient's body.

FIG. 6 further shows connectors 121 of fastening means 120 (not shown) which enable fixing of the device at a desired location on a patient's body. The connectors 121 comprise hooks 122 or other means for enabling engaging, as will be explained later below.

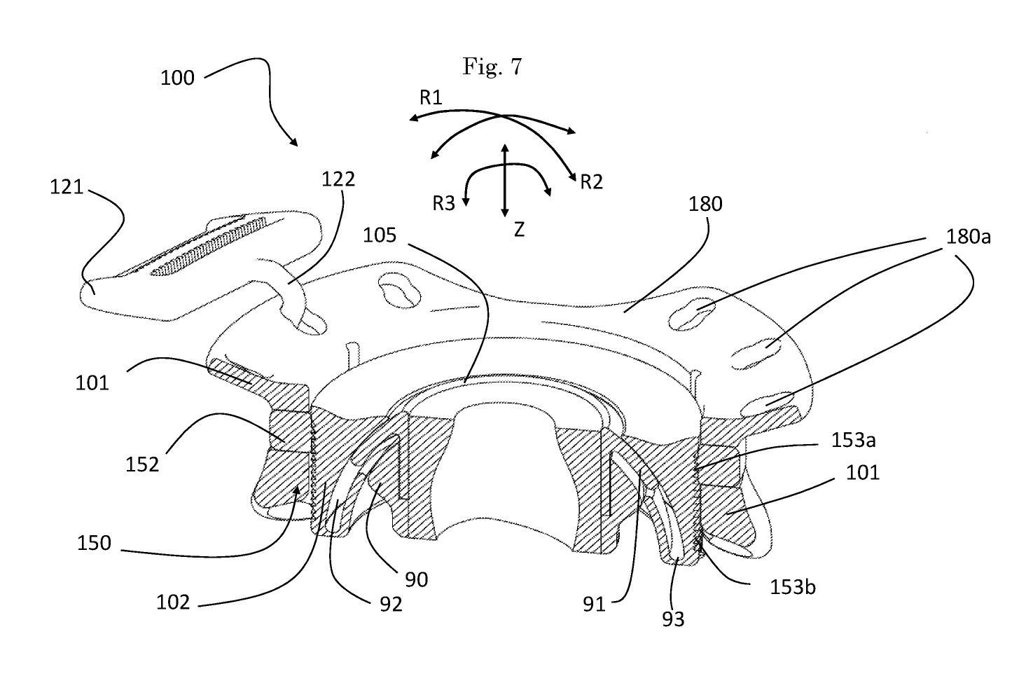

FIG. 7 shows an embodiment of the device 100, comprising a base 101 movable connected to a base module 102. The base module 102 is mounted on the base 101, wherein the base module 102 is arranged for holding the receiver module 105 and the receiver 106. The base module 102 comprises a base cavity for receiving and holding the receiver module 105. The receiver 106 of the receiver module 105 is arranged for receiving and retaining the ultrasound probe 200 and comprises an opening formed therethrough. The receiver 106 is customizable to the ultrasound probe 200, so that different probes having different dimension can be used by the same device 100. Advantageously, the receiver 106 is modular and arranged for being interchangeable for a particular ultrasound probe 200.

A lockable ball and socket connection provides a simple and reliable way for orientation of the receiver module 105 with respect to the base module 102. The receiver module 105 has at least partially a spherical/spheroid shape (cf. ball), with flattened upper and lower portions. The base module 102 has curved walls forming a portion of a spherical shape, wherein the curved walls are shaped to receive an outer surface of the receiver module 105. The receiver module 105 can be arranged to at least partially have a spherical outer shape such as to fit snugly within the base module 102. Such a ball and socket connection allows the device 100 to hold the receiver module 105. Therefore, when in use, the ultrasound probe 200 can be received and positioned by the device 100 to extend through an opening of the receiver module 105 so as to be held by the receiver 106 and orientated relative to a patient's body. The receiver module 105 having a spherical outer shape can be enclosed by the base module 102 and accordingly be rotated as appropriate to establish a desired orientation of the device 100 relative to the body. The device 100 is configured such that the orientation can be changed while the probe 200 is inserted in the device 100 and pushed against the body of the patient.

The locking means allow the receiver module 105 to be locked within the base module 102 so as to prevent further rotation of the ball and so as to hold the receiver module 105 substantially stationary with respect to the base module 102. In this way, a certain desired orientation of the receiver module 105 can be maintained. For example, pushing members can be employed which can urge against the receiver module 105 increasing the pressure and resistance for movement of the receiver module 105 within the base module 102. In an example, the sliding surfaces between the base module 102 and the receiver module 105 can be coated with a frictional material which allows to use a lower pressure for obtaining a locked state. Alternatively, the surfaces can be coated or smoothened to reduce the friction so that movement during orientation is facilitated.

In the shown embodiment, the receiver module 105 comprises two spherical walls, namely an inner spherical wall 90 and an outer spherical wall 91, wherein the outer spherical wall 91 forms a sleeve 91 which is arranged for being movable inside a spherical guide opening 92 of the base module 102. The range of motion of the receiver module 105 relative to the base module 102 is restricted by the sleeve 91 of the receiver module 105 encountering a stop 93 arranged at an end portion of the spherical guide opening 92. Such a connection between the base module 102 and the receiver module 105 enables rotation of the receiver module 105 around a plurality of axes of rotation R1, R2, and R3, such that a combined rotation can be achieved.

In an embodiment, the opening of the base module 102 is shaped to restrict the range of motion of the receiver module 105. The opening of the base module 102 is smaller than an effective cross-sectional dimension of the receiver module 105, forming a restricted opening. If a force is applied to the receiver module 105 along a longitudinal axis of the shaft, the force can be absorbed by a bearing surface area of the base cavity.

If the receiver module 105, being enclosed by the base module 102, is oriented properly and held in rotatable contact with the base module 102, the base module 102 can be advanced inward with respect to the base 101 by adjustment of the height so that an inserted ultrasound probe 200 held by the device 100 can be urged against the body of a patient. The base 101 is arranged for providing a contact interface at a surface contact area between the device 100 and the patient. Such a contact interface is obtained when the device is in use. Furthermore, when an ultrasound probe 100 is inserted in the receiver 106, the probe 100 can be held in contact with the patient's body, forming a second contact interface with a patient's body. The contact pressure exerted by the probe head 35 on the patient's body can be influenced by adjusting the height of the probe 200 with respect to the patient's body. For this purpose, the relative displacement between the base 101 and the base module 102 is adjusted so that the receiver 106 and hence the probe 200 can be moved towards the patient's body along a longitudinal axis Z of the device 100. When the probe is positioned as desired, also the height can be locked by the height locking means 150 so that the probe 200 can be tightly pushed against the patient's body.

In the shown embodiment, the height locking means 150 are arranged for providing discrete locking positions in which the distance between the receiver 106 and the surface contact area can be fixed. The locking means 150 can provide a fixed or locked connection between the base module 102 and the base 101 so that displacement of the base module 102 relative to the base 101 in a longitudinal direction, can be prevented. The locking means 150 comprise an engaging member 152 which can be moved to a locked position and an unlocked position, wherein in the locked position the engaging member 152 engages the base module 102 so as to prevent movement of the base module 102 with respect to the base 101, and wherein in the unlocked position the engaging member 152 releases the base module 102 so as to enable movement of the base module 102 with respect to the base 101. The movement can be prevented as the movement of the engaging member 152 of the locking means 150 is constrained in the longitudinal direction of the device 100. In this example, the engaging member 152 is arranged in a groove and is only movable in a radial direction (i.e. transversal with respect to a longitudinal direction of the device 100) so as to switch between the locked position and unlocked position.

An adjustment of the distance between the receiver 106 and the surface contact area allows a height adjustment of the receiver 106 relative to the surface contact area. The receiver 106 can therefore be fixed at the adjusted distance/height by the height locking means 150 so that the contact pressure or pressing force of the ultrasound probe 200 on a patient's skin can be adjusted. This allows the operator of the ultrasound probe 200 to obtain more control over the measurements. A distance between the receiver 106 and the surface contact area can be seen as a height of the receiver. In this embodiment a locking position by the engaging member 152 of the locking means 150 is achieved by a plurality of protrusions and indentations 153a (saw-tooth shaped) engaging a plurality of complementary protrusions and indentations 153b (saw-tooth shaped) arranged on a portion of the outer surface of the base module 102. In this example, the engaging protrusions and indentations are arranged on two opposite sides of the device 100 so as to improve the clamping, reliability and stability of the locking means 150. Many variants are possible. In an example, the engaging member comprises a single protrusion which can be inserted in an indentation arranged on a portion of the outer surface of the base module 102 so as to provide a locking engagement between the base module 102 and base 101. In an example, the engaging member comprises a relatively large indentation in which a relatively large protrusion arranged on a portion of the outer surface of the base module 102 can be inserted for obtaining a locking engagement.

In a further example, the device 100 further comprises a threaded connection between the base module 102 and the base 101 so that a height adjustment can be achieved by a rotation of the base module 102 with respect to the base 101. Other alternatives can be envisaged, such as a railed connection, sliding connection, etc.

In this embodiment, the base 101 further comprises configurable fastening member 180 providing selective fastening positions 180a at which one or more fastening means 120 (e.g. straps, such as illustrated in FIG. 1) can be attached. The fastening means 120 may for example comprise connectors 121 comprising hooks or pins 122 that enable to engage at one or more of the fastening positions 180a. In this embodiment, the configurable fastening member 180 comprises three selectable fastening positions 180a. Other series are possible, such as four, five, ten, etc. The fastening positions 180a are provided by through holes inside the configurable fastening member, wherein the hooks 122 may be inserted. Other solutions are possible, for instance using a rail. It is advantageous to employ mechanisms which reduce the risk of dirt accumulation, for instance due to small cavities, narrow trenches, unreachable indentations, etc., so as to reduce the risk of contamination as a result of the use of the device in a medical environment. As illustrated in FIG. 7, the through holes (or similar implementations) at the fastening positions 180a allow fastening belts (or straps or other means) comprising connector pins, hooks or other connectors 122 to engage with the base 101. In this way, the receiver 106 is easily vertically displaceable and can be fixed by locking means 105 at a certain configurable distance of the receiver 106 to the surface contact area.

The height locking means 150 can be arranged such that the engaging member 152 is biased towards a locked position. For example, when the operating parts of the handle 150a as shown in FIG. 6 are pushed towards each other, the locking means 150 can release the engaging member 152 from the base module 102. If the pushing of the operating parts of the handle is halted, the engaging member 152 will be biased towards the base module 102 so as to form a locking engagement, i.e. locked position.

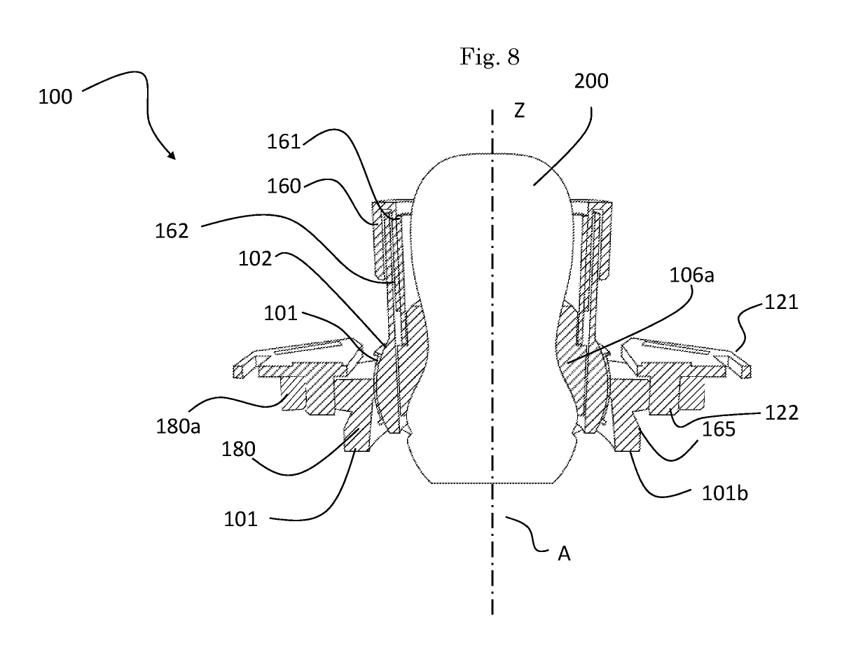

FIG. 8 shows an embodiment of the device 100 holding an ultrasound probe 200. The device 100 is arranged such that the height of the receiver 106 is adjustable. For the height adjustment of the receiver 106, the distance of the receiver 106 to the surface contact area 101b is adjustable in a longitudinal direction Z of the device 100. Furthermore, the receiver 106 is easily positionally adjustable by a rotatably adjustable ball and socket type connection (ball joint). The device 100 comprises a cup-shaped base 101 and a complementary base module 102 arranged to be movable inside the cup-shaped base 101. The base 101 can further have a horse-shoe shape (not shown), for facilitating coupling of the base module 102 to the base 101. In the embodiment of FIG. 8, the height adjustment mechanism is different compared to the height adjustment mechanism shown in the embodiments of FIGS. 6 and 7. In the shown embodiment of FIG. 8, the device 100 comprises a height adjustment ring 160 which can be operated for adjusting its height. In this embodiment, the height can be adjusted by rotation of the height adjustment ring 160. Advantageously, the height adjustment ring 160 can also be used for manipulation of the orientation of the base module 102 by a user. In this way, the user can tilt the ultrasound probe by grabbing and moving the height adjustment ring 160. The device further comprises an elevator 161 which is arranged for moving the receiver 106 up and down in a longitudinal direction of the base module 102. The receiver 106 comprises a fitting 106a which is configured to form an connection adapter between the device 100 and the ultrasound probe 200. In this embodiment the fitting 106a slots into the height adjustment ring 160 from below in the direction of the device 100, i.e. direction A. In this way, the fitting 106a can be appropriately held in position when the ultrasound probe 200 is pushed against the skin of the patient. The fitting 106a can be a one-pieced hinged elastomer.

The ball and socket type connection can be configured to allow tilting of the base module 102 (and hence also the receiver 106 and fitting 106a) over +20°/−20°. Other configurations are possible, such as for example +10°/−10°, +45°/−40°, etc.

The base 101 further comprises supporting means with three contact legs 165 (one shown in FIG. 8). The contact legs 165 are integrated with the base 101. However, in an alternative embodiment, the supporting means may also be removable coupled to the base 101.

The base 101 of the device 100 further comprises a fastening member 180 providing at least one fastening positions 180a at which one or more fastening means 120 can be attached for achieving fixation. The device 100 can be secured to a patient by means of one or more straps and strap clips. For example, a horizontal cross-chest straps, shoulder straps, knee straps, arm straps, etc. Other types of straps can be used. The fastening member 180 may be removable connected to the base 101. Furthermore, in an example, the fastening member 180 is disposable after use.

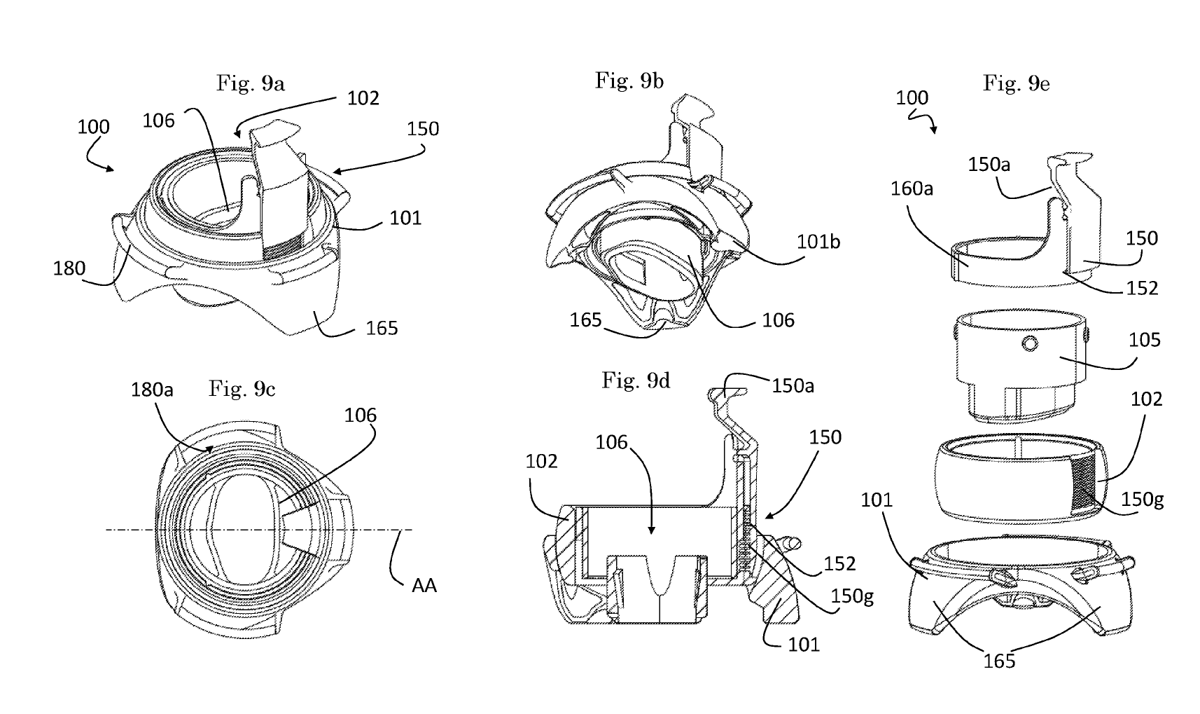

FIGS. 9A-9E show another embodiment of the device 100 according to the present disclosure. The device 100 comprises a base 101 and a base module 102 including a receiver module 105 and a receiver 106. FIG. 9A shows a perspective view of an upper side of the device 100. The receiver 106 is customizable to the ultrasound probe 200 to be held by the device 100. Further, the base module 102 and/or the receiver module 105 can be modular and arranged for being interchangeable for a particular ultrasound probe 200 used with the device 100. FIG. 9B shows a perspective view of a bottom side of the device 100. The receiver 106 has an insertion opening forming a through hole into which an ultrasound probe 200 can be received. The receiver 106 can be arranged and customized for one or more types of ultrasound probes 200 having particular dimensions and shapes. Since the receiver 106 is customizable, different types and models of ultrasound probes 200 can be employed and used with a single holder device 100. This has also the benefit that an exact position and orientation of a probe held by the device 100 on the body of a patient can be used to perform other measurements with another probe 200, wherein only the probe 200 and/or receiver 106 is changed.

The base module 102 is rotatably connected to the base 101. Furthermore, the base module 102 is displaceable arranged with respect to the base 101 so that its height can be adjusted and locked by means of height locking means 150.

The base 101 of the device 100 comprises three fastening members 180 each providing a plurality of fastening positions 180a at which one or more fastening means 120 can be attached for achieving fixation. Therefore, the fastening member 180 is configurable providing selective fastening positions 180a at which one or more fastening means 120 can be attached. The fastening member 180 comprises a rail into which the fastening means can be clamped. As a result, the user has a certain degree of freedom in the choice of the location of fixation of the fastening member 180 to the fastening means 120.

A top view of the device 100 is shown in FIG. 9C. A cross-sectional view along the shown axis AA in FIG. 9C is shown in FIG. 9D. An exploded view of the embodiment of the device 100 is shown in FIG. 9E.

As shown in FIG. 9C, the receiver 106 can be configured and shaped to receive an ultrasound 200 within a range of acceptable dimensions and shapes. However, as the receiver 106 is removably connected to the device 100, it can be replaced if required, so that also other ultrasound probes 200 can be used by the device. In this way, the device 100 can be customized to an ultrasound probe 200. The receiver 106 can be made of a substantially rigid material or a substantially flexible material. Combinations are also possible, for instance a receiver 106 having one or more substantially rigid portions (e.g. ribs) enclosing one or more substantially flexible cushions arranged on an inner side of the insertion opening for being in contact with the ultrasound probe 200. In this way, a tight grip of said probe can be achieved.

The height locking means 150 provide a plurality of discrete locking positions in which the distance between the receiver 106 and the surface contact area can be locked. Note that in this locked position, the base module 102 can still be tilted and orientated with respect to the base 101. Furthermore, the receiving module 105 is displaceable with respect to the base module 102, in a longitudinal direction of the base module 2. The base module 2 has a ring-shaped body with a spherical outer surface which conforms with a spherical inner surface of the base 101 so as to allow a tilting connection between the two. In this way, the orientation of the base module 102 with respect to the base 101 can be adjusted, hence also the orientation of the receiver module 105 which is connected by the base module 102 can be changed. The locking means 150 of the device 100 is arranged such that a locked connection between the base module 102 and the receiving module 105, wherein displacement along a longitudinal axis of the base module 102 is prevented. The locking means 150 comprise an engaging member 152 which can be moved to a locked position and an unlocked position, wherein in the locked position the engaging member 152 engages the base module 102 so as to prevent movement of the base with respect to the receiver 105. In the unlocked position, the engaging member 152 releases the base module 102 so as to enable movement of the base module 102 with respect to the receiver module 105 and thus also the receiver 106 which is carried by the receiver module 105. The movement can be prevented as the movement of the engaging member 152 of the locking means 150 is constrained in the longitudinal direction of the base module 102. In this example, the engaging member 152 comprises one tooth 152 which is arranged to fit into one of a plurality of grooves 150g arranged on portion of the base module 102. The tooth 152 can be moved inside a groove 150g for establishing a height locking position, and can be moved outside of the groove 150g so as to release the height locking position. A plurality of grooves 150g can be arranged so as to obtain a plurality of height locking positions. The tooth 152 is removed from a groove by moving said tooth in a radial direction, i.e. transversal with respect to a longitudinal direction of the base module 102. Then, the receiver module 105 becomes axially displaceable with respect to the base module 102. The height locking means 150 comprise a handle 150a for releasing the locking means 150, i.e. moving the engaging member 152 (tooth) from a locked state to an unlocked state. Alternative locking mechanism are envisaged.

An adjustment of the height of the receiver module 105 may influence the contact pressure or pressing force of the ultrasound probe 200 on a patient's skin, which may result the data obtained by the measurement instrument 200.

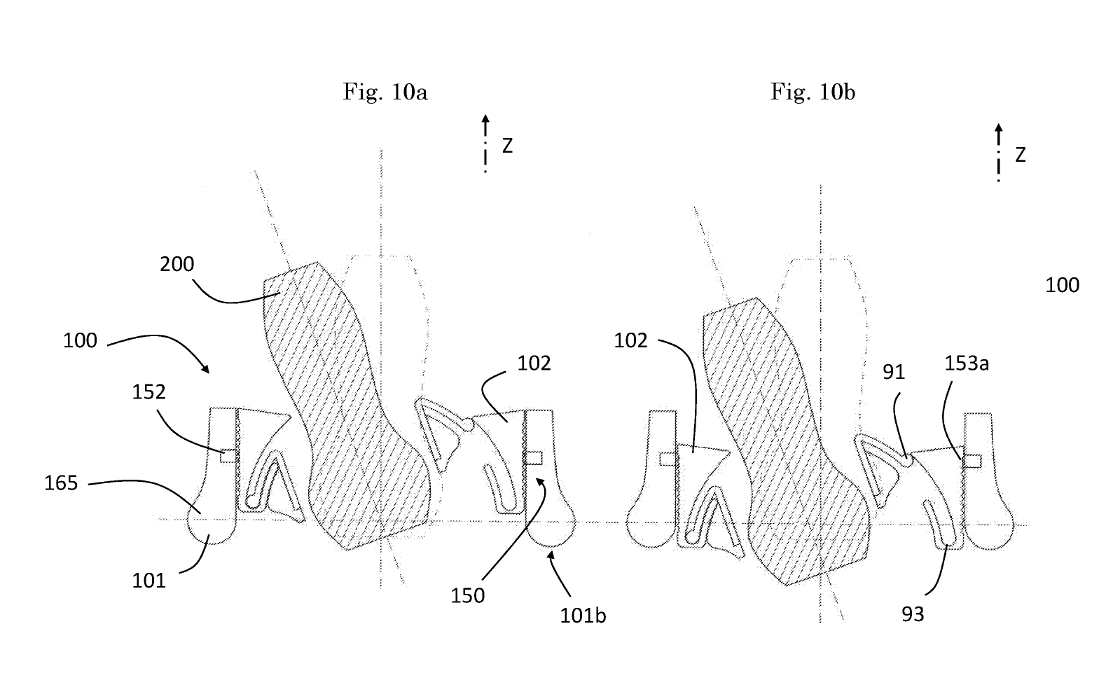

FIGS. 10A and 10B show a cross-sectional view of an embodiment of the present invention. The device 100 is arranged for holding an interchangeable ultrasound probe 200 for medical application. The device 200 comprises a base 101 which can be coupled to fixation means arranged for fixating the device 100 at a surface contact area 101b of the device 100 to a position on a patient's skin. Further, a base module 102 is arranged for being connected to the base 101 and arranged for holding the ultrasound probe 200. The base module 102 further comprises adjusting means 150 arranged for adjusting the position of the ultrasound probe 200 held by the base module 102. The adjusting means 150 comprises a locking mechanism 152 arranged for fixating the adjusted position of the ultrasound probe 200. The base module 102 comprises a receiver 106 arranged for receiving and retaining the ultrasound probe 200. The receiver 106 is customizable to the ultrasound probe 200.

The base module 102 and/or receiver 106 can be modular and arranged for being interchangeable for a particular ultrasound probe 200.

A bottom side of the legs 165 of the base 101 comprise a contact surface area 101b configured to be brought into contact with a patient's skin when using the device 100. Ideally, parts of the device that are to be brought in contact with a patients skin, are designed such as to be easily cleanable so that they may be sterilized for hygienic purposes. In accordance with embodiments, such parts are disposable or comprise removable disposable parts or disposable covers or sleeves, which make sterilization superfluous for ease of use. For example, the contact legs 165 may comprise disposable portions at their distal ends where these are to be in contact with the patient's skin.

The distance of the receiver 106 to the surface contact area can be adjusted in a longitudinal direction of the device 100. The base module 102 is movably connected to a base 101 so that the distance between the base module 102 relative to the surface contact area can be adjusted. Furthermore, the device 100 comprises a height locking means 150 arranged for locking the longitudinal distance of the receiver 106 with respect to the surface contact area. The height locking means 150 are configured to provide discrete locking positions in which the distance between the receiver 106 and the surface contact area can be fixed.

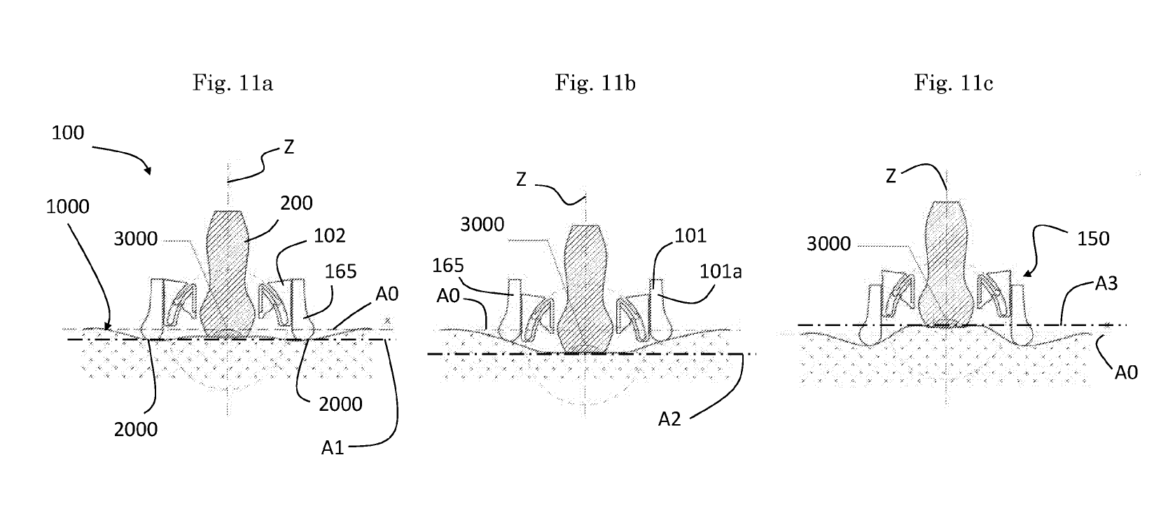

FIGS. 11A, 11B, and 11C show a cross-sectional view of an embodiment of the present invention. The device 100 is displaced along a longitudinal direction of the device 100. The base 101 which is connected to the base module 102 comprises legs 165 which are brought in contact with a patient's skin 1000.

In the shown embodiment, the base is arranged for providing a contact interface with a patient's body, namely at the skin-device contact locations 2000. The base module 102 is arranged for carrying the receiver module 105 which comprises a receiver arranged to receive and retain an ultrasound probe 200. Further, the base module 102 is arranged to be displaceable with respect to the base 101 such that the relative displacement between the base module 102 and the base 101 in a longitudinal direction of the device, base 101 and/or base module 102 is adjustable, namely along axis Z in this embodiment. Note that in an embodiment, the axis may also be configured to follow relative orientation and/or tilting of the base module 102.

The device 100 comprises three legs 165 with rounded distal ends so as to improve contact with the patient. Advantageously, the device can be secured in a more stable fashion using the three contact legs of which the contact locations form a triangle on a plane. The contact legs 165 are rounded such as to more evenly distribute the contact pressure exerted by the device 100 onto the body of the patient at skin-device contact locations 2000. An initial height of the patient's skin 1000 is illustrated by a reference line A0. The device 100 is pushed against the patient's skin 1000 and held in such a condition by means of fixation means, which are arranged for fixating the device 100 at a surface contact area of the device to a position on a patient's skin 1000. As a result of this, the height of the patient's skin 1000 is changed from the reference line A0 to a first skin-probe contact line A1, as shown in FIG. 11A. Height adjustment of the device 100 can influence the device-skin-interaction. In FIG. 11B, the base module 102 is displaced with respect to the base 101, which is in contact with the patient's skin 1000 by means of contact legs 165. The base module 102 is moved longitudinally in the direction of the patient's skin 1000 corresponding to a longitudinal direction of the base module 102 and device 100. The ultrasound probe 200 is pushed further (deeper) against the skin 1000 at a skin-probe contact location 3000, resulting in a second skin-probe contact line A2 which is situated under the reference line A0 and lower compared to the first skin-probe contact line A1. In FIG. 11C, the base module 102 is moved upwards away from the patient's skin 1000, i.e. in a longitudinal direction away of the patient's skin 1000 corresponding to a longitudinal direction of the base module 102 and device 100. In this case, the ultrasound probe 200 is backed away from the patient's skin 1000 while maintaining contact. The skin 1000 between the contact legs 165 can be pushed up towards the ultrasound probe 200 and remain in contact herewith. As a result the skin-probe contact location 3000 is pushed upwards and is located above the skin-device contact locations 2000. This results in a third skin-probe contact line A3 which is situated above the reference line A0. The skin contact interaction can be manipulated by adjusting the height of the base module 102 and/or receiver module 105 holding the ultrasound probe 200.

Certain members of most embodiments of the present invention can be made in multiple parts designed for modular assembly of different sizes and shapes and for easy removal and, if necessary replacement of some members or parts of members without disassembly of the entire assembly. Any of such parts, or even the whole device, may be made disposable to obviate the need of sterilization of such parts to prevent inter-patient contamination or infection. Next to the receiver module 105, receiver 106, fitting 106a, the removable parts may include for example the base module 102, base 101, contact legs 165, a distal part of contact legs 165, fastening member 180 and locking mechanism 150 of the device 100. Other parts can also be removable. Regarding the use of disposable parts with any of the embodiments of the present invention, it is noted that in general, any part or parts of the device that make contact with a patient's skin may be made removable and/or disposable, or may be provided with disposable sleeves or covers.

A plurality of probes can be fixated on a patient's body using one or more devices 100. This may allow simultaneous measurement at different measurement locations, i.e. locations in which the probe is held by the device 100. Advantageously, three dimensional images may be built using data retrieved by two or more probes. Therefore, in an embodiment, the device 100 can comprise a plurality of receivers in for receiving and holding an ultrasound probe. However, it may also be possible to use two or more separate devices 100 each comprising one receiver, wherein the relative orientation of the devices 100 with respect to each other are taken into account for building a three-dimensional image from measurement data of the probes held by the devices 100.

In an embodiment, the rotation, tilting, and/or displacement of the receiver for the ultrasound probe may be adjusted using an actuator (e.g. motor), which can be remotely controlled by the user. Embodiments configured for allowing both manual movement and motorized movement are also envisaged.

In order to get a good image with the ultrasound probe, the head 35 needs to be connected with the skin without any air in between. For this reason a ultrasound gel on water basis is used. This is done before the procedure, but in some cases need to be added during the procedure. For this purpose, in a further embodiment, the device may comprise a channel or cavity that may be used for adding or conveying ultrasound gel to the area where the head of the probe is to make contact with the patient's skin. This cavity is constructed in such a way that the gel can be added at moments that the device 100 is displaced without the head 35 coming of the skin completely and there is no space to add the gel sideways between the legs 101.

It is thus believed that the operation and construction of the present invention will be apparent from the foregoing description. The invention is not limited to any embodiment herein described and, within the purview of the skilled person. Modifications are possible which should be considered within the scope of the appended claims. Equally all kinematic inversions are considered inherently disclosed and to be within the scope of the present invention. The term comprising when used in this description or the appended claims should not be construed in an exclusive or exhaustive sense but rather in an inclusive sense. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. Features which are not specifically or explicitly described or claimed may be additionally included in the structure according to the present invention without deviating from its scope.

It should be noticed that this invention is relatively easy to produce and that even the cost linked to implementing the invention is not very high. The invention described above may be modified and adapted in several ways without thereby departing from the scope of the inventive concept. Moreover, all details of the invention may be substituted with other technically equivalent elements and the materials used, as well as the shapes and dimensions of the various components, may vary according to requirements.

References to “one embodiment”, “an embodiment”, “example embodiment”, “various embodiments”, etc., indicate that the embodiment(s) of the invention so described may include particular features, structures, or characteristics, but not every embodiment necessarily includes the particular features, structures, or characteristics. Further, some embodiments may have some, all, or none of the features described for other embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical. However, “coupled” may also mean that two or more elements are not in direct contact with each other, but yet still cooperate or interact with each other in some way.

The description may use perspective-based descriptions such as horizontally, vertically, height, depth, up, down, back, front, top, bottom, side, longitudinally, axially, etc. Such descriptions are merely used to facilitate the discussion and are not intended to restrict the application of disclosed embodiments.

Many variants will be apparent to the person skilled in the art. All variants are understood to be comprised within the scope of the invention defined in the following claims.

1. A device for holding an interchangeable ultrasound probe for medical application comprising:
a base comprising a fixation means arranged for fixating the device at a surface contact area of the device to a position on a patient's skin;
a base module arranged for being connected to the base and arranged for holding the ultrasound probe;
the base module further comprising adjusting means arranged for adjusting the position of the ultrasound probe; and
the adjusting means comprising a locking mechanism arranged for fixating the adjusted position of the ultrasound probe;
wherein
the base module comprises a receiver arranged for receiving and retaining the ultrasound probe; and
wherein the receiver is customizable to the ultrasound probe.

2. The device according to claim 1, wherein at least one of the base module and receiver are modular and arranged for being interchangeable for customizing the receiver for a particular ultrasound probe.

3. The device according to claim 1, wherein the base module is arranged for being displaced in a rotational direction from a first rotated position to a second rotated position in a plane of the surface contact area.

4. The device according to claim 1, wherein the base module is arranged for being displaced in fixed steps.

5. The device according to claim 1, wherein the base comprises a ring having a first raised wall and the base module comprises a ring having a second raised wall.

6. The device according to claim 1, wherein the base module is arranged for being tilted from a first tilted position to a second tilted position compared to a plane of rotation.

7. The device according to claim 1, wherein the locking mechanism comprises a configuration of the group of configurations comprising:
a configuration for locking the base module in a rotated position, wherein multiple inner longitudinal recesses are comprised in the first wall, said recesses positioned substantially perpendicular to the plane of the surface contact area, and wherein said recesses are arranged for receiving at least one protrusion arranged on the outside of the second wall;
a configuration for locking the base module in a rotated and/or tilted position, wherein the base module comprises a locking element which is arranged for at least one locking and unlocking rotation of the base module by manipulating the locking element; and
a configuration for locking the base module in a at least one of a rotated and tilted position, wherein the base module comprises a friction element exerting a default level of friction force for holding the base module in a first tilted position, and which is displaced to a second tilted position by manual power overcoming the default level of friction force.

8. The device according to claim 7, wherein the base module comprises a rotatably connected tilt member, said tilt member comprising the at least one protrusion.

9. The device according to claim 1, wherein the receiver comprises a fitting arranged for being fit into the receiver and which comprises a through hole arranged for inserting or removing the ultrasound probe.

10. The device according to claim 1, wherein the material surrounding the through hole of the fitting comprises a resilient material.

11. The device according to claim 1, wherein the base is arranged to be displaceable with respect to the base module such that the relative displacement between the base and the base module in a longitudinal direction of the base and/or base module is adjustable.

12. The device according to claim 1, wherein a distance of the receiver to the surface contact area is adjustable in a longitudinal direction of the device.

13. The device according to claim 11, wherein a height locking means is arranged for locking the longitudinal distance of the receiver with respect to the surface contact area.

14. The device according to claim 13, wherein the height locking means is configured to provide discrete locking positions in which the distance between the receiver and the surface contact area is fixed.

15. The device according to claim 1, wherein the fixation means comprises at least one strap, said strap comprising an element of a group comprising:
an elastic band comprising at least one part for surrounding a body part of the patient and arranged for being fastened to the device or to another part of the elastic band; and
a non-elastic strap comprising at least one part for surrounding a body part of the patient and arranged for being fastened and tightened to the device or to another part of the non-elastic strap.

16. The device according to claim 1, wherein the device further comprises supporting means arranged for providing a contact interface between the device and a patient's skin, wherein the supporting means comprises at least three contact legs.

17. The device according to claim 16, wherein the supporting means comprise contact legs having one or more removable and/or disposable portions.

18. The device according to claim 16, wherein each of the contact legs comprises a base and a distal end, the base arranged for being coupled with the device, the distal end having a surface contact area configured for being in contact with a patient's skin so as to provide support for the device, wherein the distal end is pivotally connected with the base.

19. The device according to claim 1, wherein the device further comprises one or more configurable fastening members providing a plurality of selective fastening positions at which one or more fastening means are attached.

20. A method for holding an interchangeable ultrasound probe for medical application comprising: a base comprising a fixation means arranged for fixating the device at a surface contact area of the device to a position on a patient's skin; a base module arranged for being connected to the base and arranged for holding the ultrasound probe; the base module further comprising adjusting means arranged for adjusting the position of the ultrasound probe,
the adjusting means comprising a locking mechanism arranged for fixating the adjusted position of the ultrasound probe, wherein the base module comprises a receiver arranged for receiving and retaining the ultrasound probe, wherein the receiver is customizable to the ultrasound probe, the method comprising: selecting an ultrasound probe with a custom size and shape, placing the ultrasound probe in the receiver, wherein the receiver is adjusted or corresponds to at least one of the size or the shape of the selected ultrasound probe such as to be customized for retaining the probe.

21. The method according to claim 20, wherein at least one of the base module and receiver are modular and arranged for being interchangeable for a particular ultrasound probe, wherein for customizing the receiver the method comprises replacing at least one of the base module and receiver so as to adapt to at least one of the size or the shape of the ultrasound probe.

22. The method according to claim 21, further comprising securing the device on a patient's body and adjusting the height of the receiver with respect to the patient's skin.
